(19) United States
(12) Patent Application Publication
(10) Pub. No.: US 2025/0082588 A1
(43) Pub. Date: Mar. 13, 2025

(54) PHARMACEUTICAL FORMULATION FOR PRESSURISED METERED DOSE INHALER

(71) Applicant: Chiesi Farmaceutici S.p.A., Parma (IT)

(72) Inventors: Enrico Zambelli, Parma (IT); Sauro Bonelli, Parma (IT); Angelo Benedetto Matturro, Parma (IT); Francesca Usberti, Parma (IT); Alessandro Cavecchi, Parma (IT)

(21) Appl. No.: 18/959,958

(22) Filed: Nov. 26, 2024

Related U.S. Application Data
(63) Continuation of application No. PCT/EP2023/064258, filed on May 26, 2023.

(30) Foreign Application Priority Data
May 27, 2022 (EP) 22175770.1

(51) Int. Cl. A61K 31/137; A61J 1/14; A61K 31/40; A61K 31/573; A61K 47/02; A61K 47/06; A61K 47/10; A61K 47/18

(52) U.S. Cl. CPC A61K 31/137; A61J 1/1468; A61K 31/40; A61K 31/573; A61K 47/02; A61K 47/06; A61K 47/10; A61K 47/183

(57) ABSTRACT

The present invention generally relates to pharmaceutcial composition comprising a LABA agent, a LAMA agent, optionally in combination with other active ingredients, a mixture of an acid and a chelating agent, a propellant and a co-solvent. The invention also provides a pharmaceutical composition for the treatment of respiratory diseases, such as asthma and COPD.

### FIELD OF THE INVENTION

The application is a continuation of International Patent Application No. PCT/EP2023/064258, filed May 26, 2023, which claims the benefit of and priority to European Patent Application No. 22175770.1, filed May 27, 2022; the entire contents of each of which are hereby incorporated by reference.

### FIELD OF THE INVENTION

The present invention generally relates to a pharmaceutical composition comprising a LABA agent, a LAMA agent, a mixture of an acid and a chelating agent, a propellant and a co-solvent; the invention further relates to the use of such pharmaceutical compositions in the treatment and prevention of respiratory diseases.

### BACKGROUND OF THE INVENTION

Pressurized metered dose inhalers (pMDIs) are well known devices for administering pharmaceutical products to the respiratory tract by inhalation. A pMDI device typically presents a medical-containing canister (or a “can” as herein referred to), and an actuator housing having a mouthpiece. The can is usually crimped with a metered valve assembly. Depending on the active ingredients and on additional components such as excipients, acids and similar, a final pMDI formulation may be in the form of a solution or a suspension. As known in the art, solution is generally intended as substantially lacking precipitates or particles, while suspension typically refers to formulation having some undissolved material or precipitates. pMDI devices may use a propellant to expel droplets containing the pharmaceutical products to the respiratory tract as an aerosol.

Glycopyrronium bromide (also known as glycopyrrolate), classified among the long-acting muscarinic antagonists (LAMA's), is a particularly efficacious bronchodilator in the treatment of respiratory diseases when in combination with LABA agents and corticosteroids.

Aerosol inhalation compositions suitable for a pMDI device comprising formoterol in combination with glycopyrronium bromide have been described in literatures.

WO 2011/076842 describes a pharmaceutical composition comprising glycopyrronium bromide dissolved in HFA propellant and a co-solvent, containing an amount of 1M hydrochloric acid (HCl) wherein the formulation shows a good stability profile.

WO 2011/076843 describes a stabilized pharmaceutical composition comprising formoterol, glycopyrronium bromide dissolved in HFA propellant and a co-solvent wherein the formulation contains an amount of 1M HCl comprised in the range 0.1-0.3 μg/μl.

WO 2015/101576 describes a pMDI device particularly suitable for the use with a formoterol, beclometasone dipropionate and glycopyrronium bromide solution, contained in a FEP coated can. As therein disclosed, the formulation contained in a FEP coated can is endowed with an improved stability and reduced amount of degradation products, mainly with regards to the N-(3-bromo)-[2-hydroxy-5-[1-hydroxy-2-[1-(4-methoxyphenyl)propan-2-ylamino]ethyl]phenyl]formamide.

The chemical stability of the active pharmaceutical ingredients (APIs) contained in the pharmaceutical compositions is particularly desirable, particularly in order to obtain formulations suitable for the market.

Although the above-mentioned prior art provides effective formulations and technical arrangements, there is still the need to find an alternative aerosol formulation comprising a LABA agent particularly in combination with a LAMA agent and a corticosteroid, that is stable over an extended product lifetime, with the possibility to use commercially available cans, such as made of aluminium or stainless steel.

We have surprisingly found that the inclusion of a mixture of an acid and a chelating agent in a formulation comprising a LAMA agent, optionally in combination with a LABA agent and/or a corticosteroid substantially avoids the degradation of said active ingredients, thus maintaining the formulation stable over an extended period, even when the formulation is contained in an aluminum canister.

Advantageously, said aerosol formulations comprising a mixture of an acid and a chelating agent as herein described, when formulated in a propellant, in the presence of a co-solvent can be usable in a pMDI device, particularly for the treatment of respiratory diseases, such as asthma and/or COPD, with excellent aerosolizing performances.

### SUMMARY OF THE INVENTION

In one aspect, the present invention refers to a pharmaceutical composition comprising a LABA agent, a LAMA agent, a co-solvent, a propellant and a mixture of an acid a chelating agent.

Particularly, the invention refers to such a formulation also comprising a corticosteroid agent.

In a further aspect, the invention refers to the use of said pharmaceutical composition comprising a LABA agent, a LAMA agent, and optionally a corticosteroid agent, a co-solvent, a propellant and a mixture of an acid and a chelating agent for use as a medicament.

In a furthers aspect, the invention further relates to the use of a pharmaceutical composition comprising a LABA agent, a LAMA agent, a co-solvent, a propellant and a mixture of an acid and a chelating agent, for the treatment and/or prophylaxis of respiratory disorders, in particular asthma and COPD.

In a further aspect, the invention refers to a canister for a pMDI device, containing the pharmaceutical composition of the invention.

In a still further aspect, the invention refers to a pMDI device containing the above indicated formulation, preferably contained in the herein described canister.

### DETAILED DESCRIPTION OF THE INVENTION

Unless otherwise defined, all technical and scientific terms used herein have the same meanings as commonly understood by the skilled in the art.

The “molar ratio” between formoterol or a salt thereof or a solvate of said salt and the acid is calculated considering the number of moles of formoterol or a salt thereof or a solvate of said salt within the formulation and number of moles of the selected acid in the formulation.

Unless otherwise indicated the term “LABA” or “LABA agent” includes in its meaning a long acting beta 2 agonist, as known in the art, such as formoterol fumarate, arformoterol, or fenoterol.

Unless otherwise provided, the term “formoterol fumarate” or “FF” refers to (R,R)-(±)formoterol fumarate or dihydrate thereof.

Unless otherwise indicated the term “LAMA” or “LAMA agent” includes in its meaning a long acting muscarinic receptor antagonist, as known in the art, such a glycopyrronium, methscopolamine, ipratropium.

Glycopyrronium bromide, chemically defined as 3-[(cyclopentylhydroxyphenylacetyl)oxy]-1,1-dimethylpyrrolidinium bromide, has two chiral centres corresponding to four potential different stereoisomers with configurations (3R,2′R)-, (3S,2′R)-, (3R,2′S)-and (3S,2′S)-. Glycopyrronium bromide in the form of any of these pure enantiomers or diastereomers or any combination thereof may be used in practicing the present invention.

Unless otherwise indicated the term “glycopyrronium bromide” refers to (3S,2′R), (3R,2′S)-3-[(cyclopentylhydroxyphenylacetyl)oxy]-1,1-dimethylpyrrolidinium bromide racemic mixture known also as glycopyrrolate (USAN name).

Unless otherwise indicated the term “EDTA” refers to ethylenediaminetetraacetic acid.

Unless otherwise indicated the term “EDTANa₄” or “tetrasodium EDTA” or “tetrasodium edetate” refers to the salt ethylenediaminetetraacetic acid with four sodium atoms.

Unless otherwise indicated the term “EDTANa₂” or “disodium EDTA” or “disodium edetate” refers a salt of ethylenediaminetetraacetic acid with two sodium atoms.

Unless otherwise indicated the term “EDTANa₂Ca” or “sodium calcium edetate” or “edetate calcium disodium” refers to a salt of ethylenediaminetetraacetic acid with two sodium and one calcium atoms.

Unless otherwise indicated the term “EDTACa” or “edetate monocalcium” refers to a salt of ethylenediaminetetraacetic acid with one calcium atom.

The term “% w/w” means the weight percentage of the component in respect to the total weight of the formulation.

The term “% w/v” means the weight percentage of the component in respect to the total volume of the formulation.

Regarding the term “apparent pH” as herein intended, it is noticed that the calculation of the pH is generally characteristic of aqueous liquid, namely where water is the dominant component. In relatively aprotic solvents (such as the propellants used in the present invention, e.g. an HFA or HFO system) protons are non-hydrated and their activity coefficients can differ from those in aqueous solution. Although the Nerst equation (describing potential of electrochemical cell as a function of concentrations of ions taking part in the reaction) with respect to electromagnetic field (EMF) applies and the pH-meter glass electrode system will generate a variable milli-volt output according to proton concentration and vehicle polarity, the pH meter reading represents the “apparent pH” according to the present invention. In this direction, the apparent pH according to the invention can be measured by technologies known in the art, as e.g. indicated in “Correlation between Apparent pH and Acid or Base Concentration in ASTM Medium” Orest Popovych, Analytical Chemistry 1964, 36, 4, 878-882; Analytical Standard Test Method (ASTM) D6423-19 “Standard Test Method for Determination of pH of Denatured Fuel Ethanol and Ethanol Fuel Blends”.

The term “chelating agent” refers to organic compounds capable of linking together metal ions to form complex ring-like structures called chelates, as e.g. indicated in Handbook of Toxicology of Chemical Warfare Agents, 2009.

As above mentioned, the present invention unexpectedly shows that the inclusion of a mixture of an acid and a chelating agent in the formulation comprising a LABA agent, optionally in combination with a LAMA agent and/or a corticosteroid, stabilizes the thus obtained formulation when contained in an aluminum can, particularly when said formulation is in the form of a solution.

According to one embodiment, the formulation of the invention is characterized by comprising a mixture of an acid selected from an organic acid, an inorganic acid or a mixture thereof, with a chelating agent. According to the present invention, the organic acids suitable for the formulation of the invention are those described e.g. in WO2019/236559.

In one preferred embodiment, the formulation of the invention is characterized by comprising a mixture of an inorganic acid and a chelating agent.

In a further preferred embodiment, the formulation of the invention is characterized by comprising an inorganic acid selected from the group consisting of: hydrochloric, nitric and phosphoric acid. Preferably the inorganic acid is hydrochloric acid (HCl). Still preferred is a mixture of hydrochloric and phosphoric acid.

In one embodiment, the formulation of the invention is characterized by comprising a chelating agent selected from the group consisting of EDTA, EDTANa₂, EDTANa₂Ca, EDTACa. Preferably the formulation comprises EDTANa₄.

In one preferred embodiment, the formulation of the invention is characterized by comprising a mixture of an inorganic acid, preferably the hydrochloric acid (HCl) and a chelating agent, preferably EDTANa₄.

In one particularly preferred embodiment, the formulation of the invention comprises a mixture of HCl and EDTANa₄. In this respect, it has been surprisingly found that a formulation suitable for pMDI administration and comprising at least a LAMA agent, and optionally a LABA agent and/or a corticosteroid, is particularly stable when a mixture of HCl and EDTANa₄ is used. From the data collected in the herein below experimental part, it is evident that the use of the mixture of HCl and EDTANa₄ provides an increase in the stability even when the formulation is contained in aluminum can. The mixture of HCl and EDTANa₄ endows the thus obtained formulation with a degree of stability in aluminum can, comparable to the stability obtainable with the FEP technology.

In a preferred embodiment, the formulation of the invention is in form of a solution.

As shown in the experimental part, Tables 2, 3, 5 and 6 the addition of a mixture of HCl and EDTANa₄ to a solution formulation comprising formoterol fumarate, glycopyrronium bromide and BDP, contained in an aluminum can, increases the stability of the formulation in terms of % residue of the active ingredients, in particular formoterol fumarate, with respect to the corresponding formulations comprising the HCl not in admixture with EDTA. As it can be appreciated said combination of inorganic acid and a chelating agent, is in fact able to stabilize not only the formoterol fumarate, but also the other active ingredients contained in the formulation, such as the glycopyrronium bromide and the beclometasone dipropionate, to such a degree which is comparable with the stability obtained by using the FEP technology.

The present invention brings several advantages to the prior art, such as the increase of the stability of the formulation over the time, good shelf life, good reproducibility of the final formulation, the maintenance of optimal chemical conditions within cans readily available in commerce, and a consistent delivery and an efficacy of medication, particularly when formulated as a solution for a pMDI device.

Even further, the mixture of an inorganic acid and a chelating agent may also avoid the use of FEP coated can, thus providing a simpler manufacturing process and final device system. As known from the prior art and as above set forth, the formulation comprising formoterol and glycopyrronium bromide contained in a FEP coated can is in fact endowed with an improved stability, not achievable when the same formulation is contained e.g. in an aluminum can.

We have now found that the combination of inorganic acid and a chelating agent, in particular the mixture of HCl and EDTANa₄, is unexpectedly able to provide a degree of stabilization of a formulation according to the present invention, when contained in aluminum can, which is comparable with the stabilization degree obtained using the FEP technology of the prior art.

According to the invention, the formulation is suitable for pMDI administration and comprises at least a LAMA agent, and optionally a LABA agent and/or a corticosteroid and a mixture of HCl and EDTANa₄.

In one embodiment, the HCl is 1M, i.e. a defined amount of an aqueous solution comprising 1M HCl is added to the pharmaceutical formulation.

In another embodiment the EDTANa₄ is added to the formulation as aqueous solution at concentration comprised between 1 and 5 mg/ml. Preferably the concentration is comprised between 2 and 3 mg/ml mg/ml.

In one embodiment, the amount of 1M HCl contained in the pharmaceutical formulation is in a range from 0.01 to 0.08% w/w. Preferably, the amount of 1M HCl is in a range from 0.010 to 0.035% w/w; more preferably the amount of 1M HCl is in a range from 0.015 to 0.020% w/w; even more preferably the amount of 1M HCl is 0.018% w/w.

In another embodiment, the amount of EDTANa₄ contained in the pharmaceutical formulation is in a range from 0.00002 to 0.002% w/w. Preferably the amount of EDTANa4 is in a range from 0.0001 to 0.0009% w/w; more preferably the amount of EDTANa₄ is in a range from 0.0001 to 0.0005% w/w; more preferably the amount of EDTANa₄ is in a range from 0.0001 to 0.0003% w/w; even more preferably the amount of EDTANa₄ is 0.0002% w/w.

In one preferred embodiment, the amount of 1M HCl contained in the pharmaceutical formulation is in a range from 0.01 to 0.08% w/w and the amount of EDTANa4 is in a range from 0.00002 to 0.002% w/w.

More preferably, the amount of HCl is in a range from 0.015 to 0.035% w/w and the amount of EDTANa4 is in a range from 0.0001 to 0.0009% w/w. Even more preferably, the amount of HCl is in a range from 0.015 to 0.025% w/w and the amount of EDTANa4 is in a range from 0.0001 to 0.0005% w/w. Still more preferably, the amount of HCl is in a range from 0.015 to 0.025% w/w and the amount of EDTANa₄ is in a range from 0.0001 to 0.0003% w/w. Particularly more preferably, the amount of HCl is 0.018% w/w and the amount of EDTANa₄ is 0.0002% w/w.

In one embodiment, the formulation of the invention is a solution comprising a LABA agent, a LAMA agent, a mixture of an inorganic acid, preferably HCl, and a chelating agent, preferably EDTANa₄, and a corticosteroid, in amounts according to the above indicated embodiments.

In one preferred embodiment, the LABA agent of the formulation according to the invention, is selected from the group consisting of: fenoterol, formoterol fumarate, formoterol fumarate dihydrate, arformoterol, carmoterol (TA-2005), indacaterol, milveterol, bambuterol, clenbuterol, vilanterol, olodaterol, abediterol, terbutaline, salmeterol, diastereoisomeric mixtures, and a pharmaceutically acceptable salt thereof or hydrate thereof.

In a further preferred embodiment, the LABA is formoterol fumarate, preferably formoterol fumarate dihydrate.

In another embodiment, the formulation of the present invention comprises salbutamol, or (R)-salbutamol (levalbuterol) or a pharmaceutically acceptable salt thereof or hydrate thereof.

Preferably, the amount of LABA according to the present invention is comprised between 0.0005-0.04% w/w, more preferably between 0.001-0.03% w/w, even more preferably between 0.005-0.02% w/w.

In one embodiment, the LAMA agent of the formulation according to the invention, is selected from the group consisting of: glycopyrronium, ipratropium, oxitropium, trospium, tiotropium, aclidinium and umeclidinium with any pharmaceutically counterion thereof.

Preferred LAMA agent is glycopyrronium bromide.

In one embodiment, the LAMA agent, preferably glycopyrronium bromide, is present in the formulation of the invention in an amount in the range from 0.005 to 0.14% (w/w), preferably from 0.010 to 0.13% (w/w), more preferably from 0.010 to 0.045% (w/w), wherein % (w/w) means the amount by weight of the component, expressed as percent with respect to the total weight of the composition.

In one embodiment, the corticosteroid component of the formulation according to the invention, is selected from the group consisting of: budesonide, beclometasone, e.g. as the mono or the dipropionate ester, flunisolide, fluticasone, e.g. as the propionate or furoate ester, ciclesonide, mometasone, e.g. as the furoate ester, mometasone desonide, rofleponide, hydrocortisone, prednisone, prednisolone, methyl prednisolone, naflocort, deflazacort, halopredone acetate, fluocinolone acetonide, fluocinonide, clocortolone, tipredane, prednicarbate, alclometasone dipropionate, halometasone, rimexolone, deprodone propionate, triamcinolone, betamethasone, fludrocortisone, desoxycorticosterone, rofleponide, etiprednol dicloacetate.

Beclometasone dipropionate (BDP) and budesonide are particularly preferred.

In a still preferred embodiment, the corticosteroid component is beclometasone dipropionate (BDP).

According to another embodiment of the present invention, the amount of the corticosteroid component, preferably BDP, is comprised between 0.01-0.7% w/w, more preferably between 0.05-0.5% w/w, even more preferably between 0.08-0.35% w/w.

In one embodiment, the present invention refers to a formulation, preferably a solution suitable for pMDI administration, comprising: a LABA agent, a LAMA agent, a corticosteroid and a mixture of an acid and a chelating agent.

In one preferred embodiment, the present invention refers to a formulation, preferably a solution suitable for pMDI administration, comprising: a LABA agent, a LAMA agent, a corticosteroid and a mixture of an inorganic acid and a chelating agent.

In a further preferred embodiment, the present invention refers to a formulation, preferably a solution suitable for pMDI administration, comprising: a LABA agent, a LAMA agent, a corticosteroid and a mixture of HCl and EDTANa₄.

In a still preferred embodiment, the present invention refers to a formulation, preferably a solution suitable for pMDI administration, comprising formoterol fumarate, glycopyrronium bromide, BDP and a mixture of an inorganic acid and a chelating agent.

In a still preferred embodiment, the present invention refers to a formulation, preferably a solution, comprising: glycopyrronium, formoterol, BDP, mixture of HCl and EDTANa₄.

As above indicated, the formulation of the invention is particularly suitable for the administration as a pMDI solution. In this respect, the present formulation also comprises a propellant and preferably, a co-solvent, as herein below described.

The propellant of the formulation according to the invention is selected from hydrofluoroalkane (HFA) and hydrofluoroolefins (HFOs) and a mixture thereof.

In one embodiment, the hydrofluoroalkane propellant is selected from the group consisting of: HFA134a (1,1,1,2-tetrafluoroethane), HFA 227 (1,1,1,2,3,3,3-heptafluoropropane, HFA152a (1,1-Difluoroethane) and mixtures thereof.

In one embodiment, the HFO propellant of the formulation according to the invention is selected from the group consisting of: 1,3,3,3-tetrafluoropropene (HFO-1234ze) and 2,3,3,3-tetrafluoropropene (HFO-1234yf).

Preferably the propellant is an HFA propellant, more preferably HFA134a.

In an equal preferred embodiment, the propellant is HFA152a.

HFAs or HFOs may be present in the formulation in an amount in the range from 75 to 95% (w/w), preferably from 85 to 90% (w/w).

According to the above described preferred embodiments, the invention refers to a formulation as above described in detail, also comprising a co-solvent and optionally a low volatile component.

Preferably, said co-solvent is a polar compound able to increase the solubility of the components within the formulation. Preferred co-solvents are aliphatic alcohols having from 1 to 4 carbon atoms, such as methanol, ethanol, propanol, isopropanol and the like, preferably ethanol, more preferably anhydrous ethanol.

When present, said co-solvent is used in an amount comprised from 5% w/w and 20% w/w, more preferably from 10% and 15% w/w.

Even if in one embodiment the formulation of the invention consists of the above indicated components, in an additional embodiment, the formulation of the invention, may optionally further comprise additional components such as excipients, additives or low volatility components. The addition of said components may be suitably calibrated in order to modulate e.g. the chemical-physical properties of the formulation.

When present, the low volatility component is a compound characterized in having a vapor pressure at 25° C. lower than 0.1 kPa, preferably lower than 0.05 kPa. Preferred low volatility components are selected from the group consisting of: glycols, propylene glycol, polyethylene glycol, glycerol or esters thereof, ascorbyl palmitate and isopropyl myristate, wherein isopropyl myristate and glycerol are particularly preferred.

In a further preferred embodiment, the present invention refers to a formulation, preferably a solution suitable for pMDI administration, comprising, consisting of or consisting essentially of: a LAMA agent, a LABA agent and a corticosteroid, a mixture of an inorganic acid and a chelating agent, an HFA propellant and an aliphatic alcohol having from 1 to 4 carbon atoms, preferably ethanol.

In a particularly preferred embodiment, the present invention refers to a formulation, preferably a solution suitable for pMDI administration, comprising, consisting of or consisting essentially of: glycopyrronium bromide, formoterol fumarate, BDP, a mixture of HCl and EDTANa₄, an HFA propellant, preferably HFA 134a or HFA 152a and ethanol, more preferably anhydrous ethanol.

In a more particularly preferred embodiment, the present invention refers to a formulation, preferably a solution suitable for pMDI administration, comprising, consisting of or consisting essentially of: glycopyrronium bromide, formoterol fumarate, BDP, a mixture of HCl and EDTANa₄, HFA 134a and ethanol, preferably anhydrous ethanol.

In an equal preferred embodiment, the present invention refers to a formulation, preferably a solution suitable for pMDI administration, comprising, consisting of or consisting essentially of: glycopyrronium bromide, formoterol fumarate, BDP, a mixture of HCl and EDTANa₄, HFA 152a and ethanol, preferably anhydrous ethanol.

In one embodiment, the present invention refers to a formulation, preferably a solution suitable for pMDI administration, comprising, consisting of or consisting essentially of: glycopyrronium bromide, formoterol fumarate, BDP, an amount of 1M HCl in a range from 0.01 to 0.08% w/w, an amount of EDTANa₄ in a range from 0.00002 to 0.002% w/w, an HFA propellant selected from HFA 134a and HFA 152a, ethanol, preferably anhydrous ethanol.

In one preferred embodiment, the present invention refers to a formulation, preferably a solution suitable for pMDI administration, comprising, consisting of or consisting essentially of: glycopyrronium bromide, formoterol fumarate, BDP, an amount of 1M HCl in a range from 0.010 to 0.035% w/w, an amount of EDTANa₄ in a range from 0.0001 to 0.0009% w/w, an HFA propellant selected from HFA 134a and HFA 152a, and ethanol, preferably anhydrous ethanol.

In a further preferred embodiment, the present invention refers to a formulation, preferably a solution suitable for pMDI administration, comprising, consisting of or consisting essentially of: glycopyrronium bromide, formoterol fumarate, BDP, an amount of 1M HCl in a range from 0.015 to 0.020% w/w, an amount of EDTANa₄ in a range from 0.0001 to 0.0005% w/w, preferably 0.0001 to 0.0003% w/w, an HFA propellant selected from HFA 134a and HFA 152a, and ethanol, preferably anhydrous ethanol.

In a particularly preferred embodiment, the present invention refers to a formulation, preferably a solution suitable for pMDI administration, comprising, consisting of or consisting essentially of: formoterol fumarate, BDP, an amount of 1M HCl of 0.024 w/w, an amount of EDTANa₄ of 0.000025% w/w, HFA 134a and ethanol, preferably anhydrous ethanol.

In an equal preferred embodiment, the present invention refers to a formulation, preferably a solution suitable for pMDI administration, comprising, consisting of or consisting essentially of: glycopyrronium bromide, formoterol fumarate, BDP, an amount of 1M HCl of 0.018 w/w, an amount of EDTANa₄ of 0.00002% w/w, HFA 152a and ethanol, preferably anhydrous ethanol.

In some embodiments, the formulation is free of further excipients other than those explicitly defined above. For instance, the formulation may be free of excipients other than the co-solvent, the propellant, the inorganic acid and the chelating agent (e.g. HCl and EDTANa₄).

According to the invention, the present formulation can be a solution, a suspension or a system comprising solution and suspension.

In a preferred embodiment, the formulation of the invention is a solution. Preferably one or more, more preferably all, of the pharmaceutically active components of the formulation, e.g. the LABA, LAMA and/or corticosteroid are completely and homogenously dissolved in the propellant and co-solvent.

As far as the can or canister is concerned, part or all of the canister of the pMDI device suitable to contain the formulation of the invention, may be made of a metal, e.g. aluminum, or metal alloys, stainless steel or anodized aluminum, fluorine passivated aluminum and the like. Alternatively, the canister may be a plastic can or a plastic-coated glass bottle.

The metal canisters may have part or all of the internal surfaces lined with an inert organic coating.

The coating is typically applied to the internal surface of the can, thus providing an internal layer acting as interface between the internal surface of the can, and the formulation therein contained.

In this regards, a suitable coated can of the invention may have part or all of its internal surfaces coated with an inert organic or inorganic coating preferably comprising: an epoxy-phenol resin, a perfluorinated polymer, a perfluoroalkoxyalkane polymer, a perfluoroalkoxyalkylene polymer (PFA), a perfluoroalkylene polymer, poly-tetrafluoroethylene polymer (PTFE or Teflon), fluorinated-ethylene-propylene polymer (FEP), polyether sulfone polymer (PES), a fluorinated-ethylene-propylene polyether sulfone polymer (FEP-PES), a polyamide, polyimide, polyamideimide, polyphenylene sulfide, plasma, mixtures or combinations thereof.

In a preferred embodiment, the invention refers to the above described formulation, contained in a pMDI canister made of aluminum or stainless steel. Thus, in one aspect, the invention refers to a pMDI canister made of aluminum or stainless steel, filled with the formulation of the invention as above described in detail. Aluminum cans are preferred.

The canister of a pMDI device is typically crimped with a metering valve for delivering a therapeutically effective dose of the active ingredients.

The metering valve assembly comprises at least one rubber gasket seal made of a proper elastomeric material selected from: low-density polyethylene, butyl or halo butyl rubbers such as chlorobutyl or bromubutyl rubbers (optionally halogenated copolymers of isobutylene with isoprene), butadiene-acrylonitrile, neoprene, EPDM (a polymer of ethylenepropylenediene monomer), TPE (thermoplastic elastomer), cycloolefin copolymer (COC) or combination thereof.

The metering valve according to the invention is typically capable of delivering a volume in the range from 25 to 150 μl, preferably in the range from 50 to 100 μl, and more preferably from 50 μl to 70 μl per actuation; the most preferred are 50, 63 and 100 μl per actuation. Suitable valves for the present invention are commercially available.

According to a further aspect of the invention there is provided a method of filling an aerosol inhaler with a pharmaceutical composition of the invention. Conventional bulk manufacturing methods and machinery well known to those skilled in the art of pharmaceutical aerosol manufacture may be employed for the preparation of large-scale batches for the commercial production of filled canisters.

As a general example said methodology may comprise the steps of:
a) preparing a solution comprising: formoterol fumarate, BDP, glycopyrronium bromide and ethanol;
b) adding the amount of 1M HCl to the ethanolic solution and mix the bulk solution;
c) adding the amount of EDTANa₄ (as aqueous solution) to the ethanolic solution and
d) filling the canister with said solution;
e) crimping with a valve and gassing with HFA propellant.

The packaged formulations of the invention are stable for extended periods of time when stored under normal conditions of temperature and humidity.

Stability is assessed by measuring content of residual active ingredient.

In a further aspect, the invention refers to the above described formulation for use as a medicament. Thus, the invention refers to the use of the formulation as herein described for the preparation of a medicament.

Preferably, the formulation of the invention is for prophylactic purposes or for symptomatic relief of a wide range of respiratory disorders, such as asthma of all types and chronic obstructive pulmonary disease (COPD).

In one preferred embodiment, the invention refers to the formulation as herein described, for the treatment and/or prophylaxis of respiratory disorders, preferably for the treatment and/or prophylaxis of asthma or COPD.

Other respiratory disorders for which use of the pharmaceutical compositions of the invention may be beneficial are those characterized by obstruction of the peripheral airways as a result of inflammation and presence of mucus, such as chronic obstructive bronchiolitis, chronic bronchitis, emphysema, acute lung injury (ALI), cystic fibrosis, rhinitis, and adult or acute respiratory distress syndrome (ARDS).

As it is will be recognized, all the herein described embodiments are to be intended as included in the scope of the present invention, also in any possible combination with all the other preferred embodiments, as herein above and below set forth.

The invention will be now described by the following not limiting examples.

### EXPERIMENTAL PART

### Example 1

A study was performed to investigate the chemical stability of formulation intended for pMDI administration comprising formoterol fumarate dihydrate (FF), glycopyrronium bromide (GB) and beclometasone dipropionate (BDP). Said formulation is a solution contained in aluminum can (Al) or in a FEP coated can (FEP) crimped with a metering valve having a 63 μl metering volume.

An amount of HCl either alone or in mixture with EDTANa₄ were added to the formulation, thus providing Formulations 1-2, as reported in Table 1.

TABLE 1

| COMPONENT | Formulation 1 % w/w | Formulation 2 % w/w |
|---|---|---|
| FF | 0.008 | 0.008 |
| BDP | 0.136 | 0.136 |
| GB | 0.017 | 0.017 |
| 1M HCl | 0.018 | 0.018 |
| EDTANa4 | 0.0002 | — |
| Ethanol anhydrous | 12 | 12 |
| HFA 134a | 87.8208 | 87.821 |

The Formulations 1-2 were put in stability chambers in inverted position at 40° C., 75% R.H. for 1 month, the API assay and relevant degradation products were measured at T1 (1 month).

The formulations were also tested at different stability conditions, at 25° C., 60% R.H. for 6 months, the API assay and relevant degradation products were measured at T3 (3 months) and T6 (6 months). APIs residue % are reported in Tables 2 and 3.

TABLE 2

| | | T1 40°/75% | | | |
|---|---|---|---|---|---|
| Formulation | Can | FF % | BDP % | GB % | Apparent pH |
| 1 | Al | 98.1 | 102.5 | 103.0 | 4.92 |
| 1 | FEP | 99.6 | 103.8 | 104.0 | 4.94 |
| 2 | Al | 92.2 | 101.9 | 103.0 | 5.51 |
| 2 | FEP | 99.2 | 101.6 | 102.3 | 4.70 |

TABLE 3

| | | T3 25°/60% | | | | T6 25°/60% | | | |
|---|---|---|---|---|---|---|---|---|---|
| | | | | | Ap- | | | | Ap- |
| Formu- | | FF | BDP | GB | parent | FF | BDP | GB | parent |
| lation | Can | % | % | % | pH | % | % | % | pH |
| 1 | Al | 97.4 | 98.5 | 100 | 5.52 | 94.2 | 100.0 | 99.0 | 5.41 |
| 1 | FEP | 100.8 | 103.2 | 104 | 5.20 | 92.2 | 98.1 | 97.0 | 5.26 |
| 2 | Al | 92.5 | 99.7 | 100 | 5.91 | 81.7 | 99.7 | 99.0 | 6.29 |
| 2 | FEP | 99.9 | 99.4 | 101.5 | 5.02 | 94.4 | 98.5 | 97.8 | 5.11 |

As it can be observed by Tables 2 and 3 when a mixture of HCl and EDTANa₄ is added according to Formulations 1, a significant improvement of the chemical stability of formoterol (FF), glycopyrronium bromide (GB) and beclometasone dipropionate (BDP) is achieved. Of note, the % FF residue may reach values higher than 90% even in aluminum can differently from the Formulation 2 wherein the % FF residue significantly decrease after 3 months in aluminum can.

The formulation 1 in aluminum can shows a significantly improved stability, in terms of FF % residue, which is comparable with the stability of the formulation in FEP coated can.

As evident from the Tables 2 and 3, the mixture of HCl and EDTANa₄ according to the invention provides a stabilization, in terms of residue % of the APIs, particularly regarding the formoterol, comparable to the high stabilization degree obtainable using the FEP technology.

### Example 2

A second study was performed to investigate the chemical stability of formulation intended for pMDI administration comprising formoterol fumarate dihydrate (FF), glycopyrronium bromide (GB) and beclometasone dipropionate (BDP) in HFA152a propellant. Said formulation is a solution contained in aluminum can (Al) or in a FEP coated can (FEP) crimped with a metering valve having a 63 μl metering volume.

An amount of HCl in mixture with EDTANa₄ was added to the formulation, thus providing Formulation 3, as reported in Table 4.

TABLE 4

| COMPONENT | Formulation 3 % w/w |
|---|---|
| FF | 0.0107 |
| BDP | 0.1788 |
| GB | 0.0224 |
| 1M HCl | 0.0240 |
| EDTANa₄ | 0.0003 |
| Ethanol anhydrous | 10 |
| HFA 152a | 89.7 |

The Formulation 3 was put in stability chambers in inverted position at 40° C., 75% R.H. for 1 month, the API assay and relevant degradation products were measured at T1 (1 month).

The formulation was also tested at different stability conditions, at 25° C., 60% R.H. for 3 months, the API assay and relevant degradation products were measured at T3 (3 months).

APIs residue % are reported in Tables 5 and 6.

TABLE 5

| | | T1 40°/75% | | | |
|---|---|---|---|---|---|
| Formulation | Can | FF % | BDP % | GB % | Apparent pH |
| 3 | Al | 96.6 | 100.6 | 101.0 | 4.48 |
| 3 | FEP | 98.6 | 102.5 | 102.5 | 4.27 |

TABLE 6

| | | T3 25°/60% | | | |
|---|---|---|---|---|---|
| Formulation | Can | FF % | BDP % | GB % | Apparent pH |
| 3 | Al | 100.4 | 102.5 | 103.0 | 4.57 |
| 3 | FEP | 99.8 | 101.3 | 102.0 | 4.50 |

As it can be observed by Tables 5 and 6 when a mixture of HCl and EDTANa₄ is added according to Formulation 3, an optimal chemical stability of formoterol (FF), glycopyrronium bromide (GB) and beclometasone dipropionate (BDP) is achieved.

The formulation 3 in aluminum can shows a significantly improved stability, in terms of FF % residue, which is comparable with the stability of the formulation in FEP coated can, especially after 3 months in aluminum can.

As evident from the Tables 5 and 6, the mixture of HCl and EDTANa₄ according to the invention provides a stabilization, in terms of residue % of the APIs, particularly regarding the formoterol, comparable to the high stabilization degree obtainable using the FEP technology.

1. A pharmaceutical composition comprising a LABA agent, a LAMA agent, a co-solvent, a propellant, and a mixture of an acid and a chelating agent.

2. The pharmaceutical composition according to claim 1, wherein the LABA agent is selected from the group consisting of: fenoterol, formoterol fumarate, formoterol fumarate dihydrate, arformoterol, carmoterol (TA-2005), indacaterol, milveterol, bambuterol, clenbuterol, vilanterol, olodaterol, abediterol, terbutaline, salmeterol, diastereoisomeric mixtures thereof, pharmaceutically acceptable salts thereof, and hydrates thereof.

3. The pharmaceutical composition according to claim 2, wherein the LABA agent is formoterol fumarate.

4. The pharmaceutical composition according to claim 2, wherein the LABA agent is formoterol fumarate dihydrate.

5. The pharmaceutical composition according to claim 1, wherein the LAMA agent is selected from the group consisting of: glycopyrronium, ipratropium, oxitropium, trospium, tiotropium, aclidinium, umeclidinium, and any pharmaceutical counterion thereof.

6. The pharmaceutical composition according to claim 5, wherein the LAMA agent is glycopyrronium bromide.

7. The pharmaceutical composition according to claim 1, wherein the acid is an inorganic acid.

8. The pharmaceutical composition according to claim 7, wherein the inorganic acid is HCl.

9. The pharmaceutical composition according to claim 1, wherein the chelating agent is selected from EDTA, EDTANa₂, EDTANa₂Ca, EDTACa, or EDTANa₄.

10. The pharmaceutical composition according to claim 1, wherein said mixture of an acid and a chelating agent is a mixture of HCl and EDTANa₄.

11. The pharmaceutical composition according to claim 1, wherein the acid is 1M HCl that is present in an amount in a range from 0.01 to 0.08% w/w.

12. The pharmaceutical composition according to claim 11, wherein the amount of 1M HCl is in a range from 0.010 to 0.035% w/w.

13. The pharmaceutical composition according to claim 12, wherein the amount of 1M HCl is in a range from 0.015 to 0.020% w/w.

14. The pharmaceutical composition according to claim 1, wherein the chelating agent is EDTANa₄, and the amount of EDTANa₄ is in a range from 0.00002 to 0.002% w/w.

15. The pharmaceutical composition according to claim 14, wherein the amount of EDTANa₄ is in a range from 0.0001 to 0.0009% w/w.

16. The pharmaceutical composition according to claim 14, wherein the amount of EDTANa₄ is in a range from 0.0001 to 0.0003% w/w.

17. The pharmaceutical composition according to claim 11, wherein the amount of EDTANa₄ is in a range from 0.00002 to 0.002% w/w.

18. The pharmaceutical composition according to claim 17, wherein the amount of 1M HCl is in a range from 0.010 to 0.035% w/w, and the amount of EDTANa₄ is in a range from 0.0001 to 0.0009% w/w.

19. The pharmaceutical composition according to claim 18, wherein the amount of 1M HCl is in a range from 0.015 to 0.020% w/w, and the amount of EDTANa₄ is in a range from 0.0001 to 0.0003% w/w.

20. The pharmaceutical composition according to claim 19, wherein the amount of 1M HCl is 0.018% w/w, and the amount of EDTANa₄ is 0.0002% w/w.

21. The pharmaceutical composition according to claim 1, further comprising a corticosteroid selected from the group consisting of: budesonide, beclometasone (BDP), BPD monopropionate ester, BDP dipropionate ester, flunisolide, fluticasone, fluticasone propionate ester, fluticasone furoate ester, ciclesonide, mometasone, mometasone furoate ester, mometasone desonide, rofleponide, hydrocortisone, prednisone, prednisolone, methyl prednisolone, naflocort, deflazacort, halopredone acetate, fluocinolone acetonide, fluocinonide, clocortolone, tipredane, prednicarbate, alclometasone dipropionate, halometasone, rimexolone, deprodone propionate, triamcinolone, betamethasone, fludrocortisone, desoxycorticosterone, rofleponide, and etiprednol dicloacetate.

22. The pharmaceutical composition according to claim 1, further comprising a corticosteroid that is budesonide or beclometasone dipropionate (BDP).

23. The pharmaceutical composition according to claim 22, wherein the corticosteroid is beclometasone dipropionate (BDP).

24. The pharmaceutical composition according to claim 1, wherein the co-solvent is an aliphatic alcohol having from 1 to 4 carbon atoms.

25. The pharmaceutical composition according to claim 24, wherein the co-solvent is ethanol.

26. The pharmaceutical composition according to claim 1, wherein the propellant is selected from hydrofluoroalkanes (HFAs), hydrofluoroolefins (HFOs), and mixtures thereof.

27. The pharmaceutical composition according to claim 26, wherein the propellant is selected from HFA134a, HFA152a, and mixtures thereof.

28. The pharmaceutical composition according to claim 27, wherein the propellant is HFA134a.

29. The pharmaceutical composition according to claim 27, wherein the propellant is HFA152a.

30. The pharmaceutical composition according to claim 1, wherein the composition is a solution.

31. The pharmaceutical composition according to claim 22, wherein the LABA agent is formoterol fumarate dihydrate, the corticosteroid is beclometasone dipropionate (BDP), the LAMA agent is glycopyrronium bromide, the propellant is HFA134a, the inorganic acid is HCl, the chelating agent is EDTANa₂, the co-solvent is ethanol, and the composition is a solution.

32. The pharmaceutical composition according to claim 22, wherein the LABA agent is formoterol fumarate dihydrate, the corticosteroid is budesonide or beclometasone dipropionate (BDP), the LAMA agent is glycopyrronium bromide, the propellant is HFA152a, the inorganic acid is HCl, the chelating agent is EDTANa₂, the co-solvent is ethanol, and the composition is a solution.

33. A canister containing the pharmaceutical composition according to claim 1, wherein the canister is made of aluminum, stainless steel, anodized aluminum, or fluorine passivated aluminum.

34. A canister for a pMDI device, containing the pharmaceutical composition according to claim 1.

35. A canister for a pMDI device according to claim 34, made of aluminum or stainless steel.

36. A pMDI device comprising a canister made of aluminum or stainless steel containing a pharmaceutical composition according to claim 1.
